(19) United States
(12) Patent Application Publication
(10) Pub. No.: US 2018/0167266 A1
(43) Pub. Date: Jun. 14, 2018

(54) Configuring MAC Parameters in Body Area Networks

(71) Applicant: Kabushlkl Kaisha Toshiba, Tokyo (JP)

(72) Inventors: Siva Kupanna SUBRAMANI, Bristol (GB); Woon Hau CHIN, Bristol (GB); Mahesh SOORIYABANDARA, Bristol (GB)

(21) Appl. No.: 15/124,771

(22) PCT Filed: Jul. 30, 2014
(86) PCT No.: PCT/GB2014/052336

§ 371 Date: Sep. 9, 2016

(51) Int. Cl. H04L 12/24; H04L 12/26

(52) U.S. Cl. CPC H04L 41/08; H04W 84/18; H04L 43/16; H04L 43/08

(57) ABSTRACT

In an embodiment, a method of medium access control in a co-ordinator node of a body area network is disclosed. The body area network comprises the co-ordinator node and a plurality of sensor nodes. Each of the sensor nodes comprises a sensor and is configured to wirelessly communicate with the co-ordinator node in time slots defined by a plurality of medium access control super-frames. The method comprises receiving at the co-ordinator node, a signal from a first sensor node of the plurality of sensor nodes, the signal comprising an indication of a measured quantity at the first sensor node, the measured quantity being indicative of an attribute of a wireless link of the body area network between the first sensor node and the co-ordinator node; estimating an attribute of the link using the indication; and configuring parameters of a medium access control superframe using the estimated attribute.

### FIELD

Embodiments described herein relate generally to medium access control in wireless body area networks.

### BACKGROUND

A wireless body area network (WBAN) is a network of sensor nodes designed for monitoring, logging and transmitting vital healthcare signals. A typical WBAN consists of multiple sensor nodes that transmit to a hub or coordinator node. The sensor nodes are extremely low powered with transmission range of only few meters. The on-body channel characteristics are challenging; fading effects can last longer (10-300 ms) than in other types of wireless network and mobility and body postures impose large shadowing effects.

The unique characteristics of the wireless channel around human body, coupled with the need for extreme energy efficiency in healthcare applications requires adaptive and configurable medium access control (MAC) protocols.

Efficient energy consumption can be achieved by optimal radio duty cycling, that is an effective listen state and a long sleep state. Radio duty cycling is performed by a MAC protocol with the aim of minimizing idle listening, overhearing and collisions; and controlling overhead that ultimately leads to power savings. The radio of a node is turned off (i.e. put in a sleep state) when the node neither transmits nor receives data thereby saving energy. Within the active period, the time slots and the access mechanism can be random access, for example contention access such as carrier sense multiple access with collision avoidance (CSMA/CA) or, scheduled access such as time division multiple access (TDMA).

The TDMA-based scheduled access approach is the most appropriate MAC solution to achieve desired energy efficiency since it avoids many common causes of energy waste such as collisions, overhearing and idle listening. Since the sensor nodes and the coordinator are synchronized in time, the sensor nodes wake up only when they have data to send to the gateway. This arrangement makes it possible to achieve more precise network synchronization between sensor nodes and the coordinator. However, due to the high volatility of WBAN wireless links, a simple static tightly synchronized TDMA schedule is inflexible under challenging scenarios.

Traditional adaptive or opportunistic scheduling approaches are not compatible with WBANs as they require that the slave nodes are continuously available for communication. This requirement is incompatible with radio duty-cycling, an energy saving mechanism that is at the core of energy-efficient protocols in WBAN.

### BRIEF DESCRIPTION OF THE DRAWINGS

In the following embodiments will be described as non-limiting examples with reference to the accompanying drawings in which:

FIG. 1 shows a wireless body area network according to an embodiment;

FIG. 2 shows a method of configuring medium access control parameters according to an embodiment;

FIG. 3 shows parameters of a medium access control which are configured in embodiments;

FIG. 4 shows a sensor node of an embodiment;

FIG. 5 shows a co-ordinator node according to an embodiment;

FIG. 6 shows the processing performed on a co-ordinator node in an embodiment;

FIG. 7 shows a method of configuring medium access control parameters according to an embodiment;

FIG. 8a shows the beacon width in regular operation in an embodiment;

FIG. 8b shows the beacon width in configuration mode in an embodiment;

FIG. 9 shows the sampling of accelerometer values for a motion estimation step in an embodiment; and

FIG. 10 illustrates the sampling of received signal strength values in an embodiment.

### DETAILED DESCRIPTION

In an embodiment, a method of medium access control in a co-ordinator node of a body area network is disclosed. The body area network comprises the co-ordinator node and a plurality of sensor nodes. Each of the sensor nodes comprising a sensor and being configured to wirelessly communicate with the co-ordinator node in time slots defined by a plurality of medium access control superframes. The method comprises receiving at the co-ordinator node, a signal from a first sensor node of the plurality of sensor nodes, the signal comprising an indication of a measured quantity at the first sensor node, the measured quantity being indicative of an attribute of a wireless link of the body area network between the first sensor node and the co-ordinator node; estimating an attribute of the link using the indication; and configuring parameters of a medium access control superframe using the estimated attribute.

In an embodiment, the attribute of the link is movement of the first sensor node.

In an embodiment, the parameters of a medium access control superframe comprise at least one of beacon width; guard time; interframe space; and acknowledgement period.

In an embodiment, the method further comprises receiving a plurality of indications of the measured quantity from the first sensor node; and determining a weighted moving average value from the received values. The attribute of the link is estimated using the weighted moving average value.

In an embodiment, configuring parameters of a medium access control superframe using the estimated attribute comprises comparing the attribute of the link with a threshold; selecting a mode based on the result of the comparison and selecting values of parameters of the medium access control superframe according to the selected mode.

In an embodiment, the mode is selected using additive increase multiplicative decrease feedback.

In an embodiment, the attribute of the link is an active period time slot. In an embodiment, configuring parameters of the medium access control superframe comprises configuring the ratio of active and inactive period of the superframe based on the active period time slot. In an embodiment, the parameters of the medium access control superframe comprise timing parameters.

In an embodiment, a computer readable carrier medium carrying computer readable instructions which when executed on a processor cause the processor to carry out a method of medium access control in a co-ordinator node of a body area network is disclosed.

In an embodiment, co-ordinator node for a wireless body area network is disclosed. The co-ordinator node comprises an antenna configured to receive signals from a plurality of sensor nodes of the wireless body area network, the signals comprising a first signal from a first sensor node of the plurality of sensor nodes, the first signal comprising an indication of a measured quantity at the first sensor node, the measured quantity being indicative of a attribute of a link of the wireless body area network between the first sensor node and the co-ordinator node; and a processor operable to estimate an attribute of the link using the indication; and configure parameters of a medium access control superframe using the estimated attribute.

In an embodiment, the attribute of the link is movement of the first sensor node.

In an embodiment, the parameters of a medium access control superframe comprise at least one of beacon width; guard time; interframe space; and acknowledgement period.

In an embodiment, the processor comprises a classifier operable to classify the state of the link based on the estimated attribute of the link.

In an embodiment, the classifier is operable to classify the state of the link y comparing the estimated attribute with a threshold.

In an embodiment, the processor is operable to configure the parameters of the medium access control superframe by selecting a mode based on the estimated attribute.

In an embodiment, a wireless body area network is disclosed. The wireless body area network comprises a co-ordinator node and at least one sensor node comprising a sensor operable measure a quantity and an antenna operable to transmit a first signal comprising an indication of the measured quantity.

In an embodiment, the sensor is an accelerometer and the measured quantity is movement.

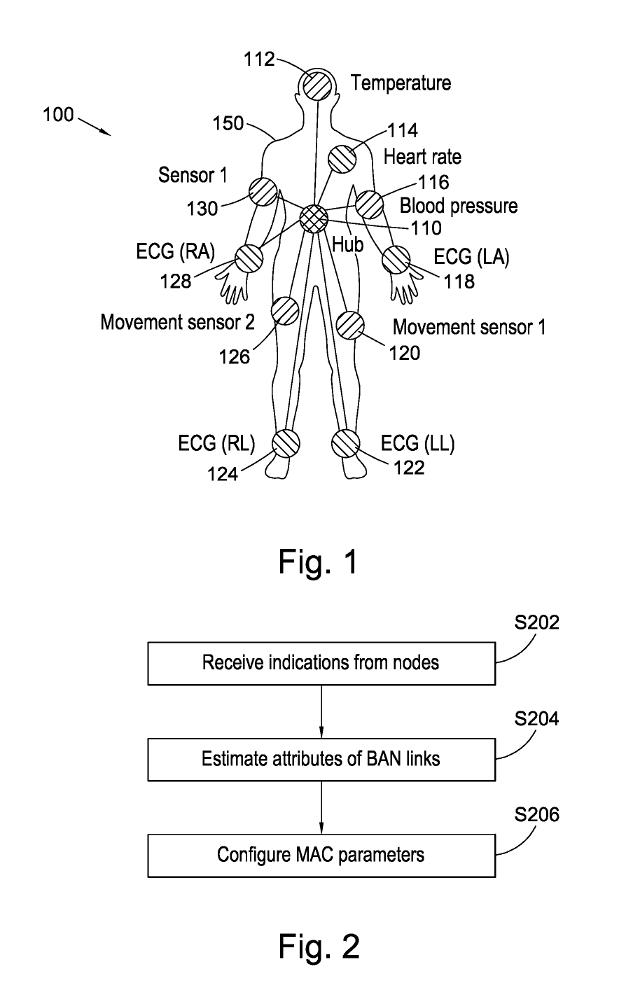

FIG. 1 shows a wireless body area network (WBAN) 100 according to an embodiment. The WBAN 100 comprises a plurality of sensor nodes which transmit sensed information to a hub or coordinator node 110. The sensor nodes are located on or implanted in the body of a patient 150 and monitor vital signs of the patient 150. The WBAN 100 comprises a temperature sensor node 112, a heart rate monitor node 114, a blood pressure sensor node 116, a left arm electrocardiograph (ECG) node 118, a first movement sensor 120, a left leg ECG node 122, a right leg ECG node 124, a second movement sensor 126, a right arm ECG node 128, and a sensor node 130.

The sensor nodes send information to the co-ordinator node 110 over the WBAN according to a medium access control (MAC) protocol which is determined by the co-ordinator node 110. The co-ordinator node 110 may connect to an external server through an off-body wireless link where the data from the sensors is stored and analysed.

Embodiments relate to the configuration of MAC parameters at a super frame level for the WBAN by the co-ordinator node. Embodiments relate to configuring the TDMA scheduled access slots and parameters at the super-frame level. Specifically, the schedule of transmissions and the associated super-frame parameters are computed by the hub and conveyed to the sensor nodes in a beacon at the start of the subsequent super-frame.

A simple static and tightly synchronized time division multiple access (TDMA) schedule is inflexible under challenging scenarios, such as a mobile or ambulatory links. In a high volatility of BAN wireless links, adaptable and configurable characteristics are necessary. Embodiments relate to methods to adaptively configure the scheduled access mode parameters, such that the MAC protocol adapts to changes in the operating environment.

FIG. 2 is a flow chart showing a method of configuring medium access control (MAC) parameters according to an embodiment. The method 200 is carried out by the co-ordinator node 110 of the WBAN 100. In step S202, the co-ordinator node 110 receives indications from the sensor nodes. The indications received from the sensor nodes indicate attributes of the wireless link between the sensor nodes and the co-ordinator node. The attributes may include a measured received signal strength indication (RSSI), information measured by the sensor of the sensor node such as an indication of movement of the node.

In step S204 the co-ordinator node 110 estimates attributes of the WBAN links from the received indications. The link attributes are one or more of the following: link quality; mobility; and application data rate. In step S206, the co-ordinator node configures MAC access parameters at the super frame level.

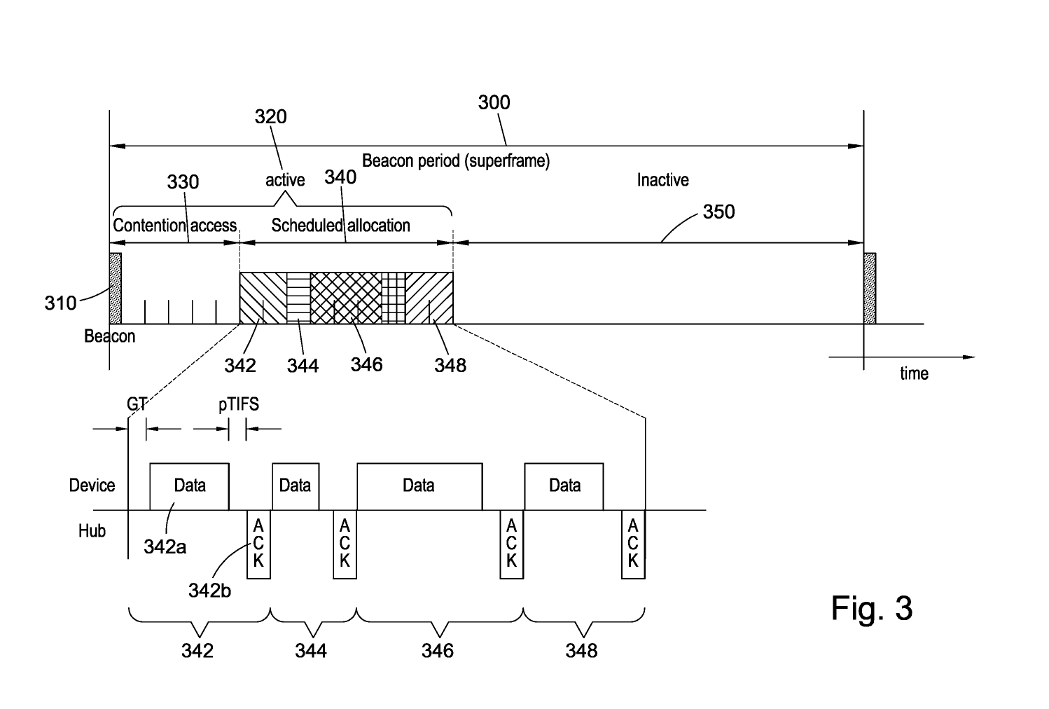

FIG. 3 shows the frame structure which is configured by a co-ordinator node according to an embodiment. The time axis is slotted and divided into periodical frames referred to as a super-frame 300. The super frame 300 consists of three parts: a beacon slot 310, an active time slot 320, and an inactive period 350.

In the beacon slot 310, the co-ordinator node 110 broadcasts the beacon signal to indicate the length of the active frame period and specific slot length allocated to each user for data transmission and acknowledgments, and the inter-frame period and parameters. In embodiments, the length of the super frame period is adaptive to the requirements of the applications.

The active period 320 has a contention access period 330 and a scheduled access period 340. In the scheduled access period 340, each of a plurality of time slots is scheduled to a sensor node where the node transmits the measured reading to the co-ordinator node (referred to as uplink). As shown in FIG. 3, a first time slot 342 is allocated to a first sensor node a second time slot 344 is allocated to a second sensor node; a third time slot 346 is allocated to a third sensor node; and a fourth time slot 348 is allocated to a fourth sensor node.

In the scheduled access period 340, in the first time slot 342, the ‘Device’ which is the first sensor node waits for a guard time (GT) before transmitting data 342a to the ‘Hub’ which is the co-ordinator node. The co-ordinator node processes the received packet of data in an inter frame spacing period (pTIFS). Following the inter frame spacing period (pTIFS), the co-ordinator node transmits an acknowledgement 342b indicating successful transmission to the first node. The process is repeated with the respective nodes for the second time slot 344, the third time slot 346, and the fourth time slot 348.

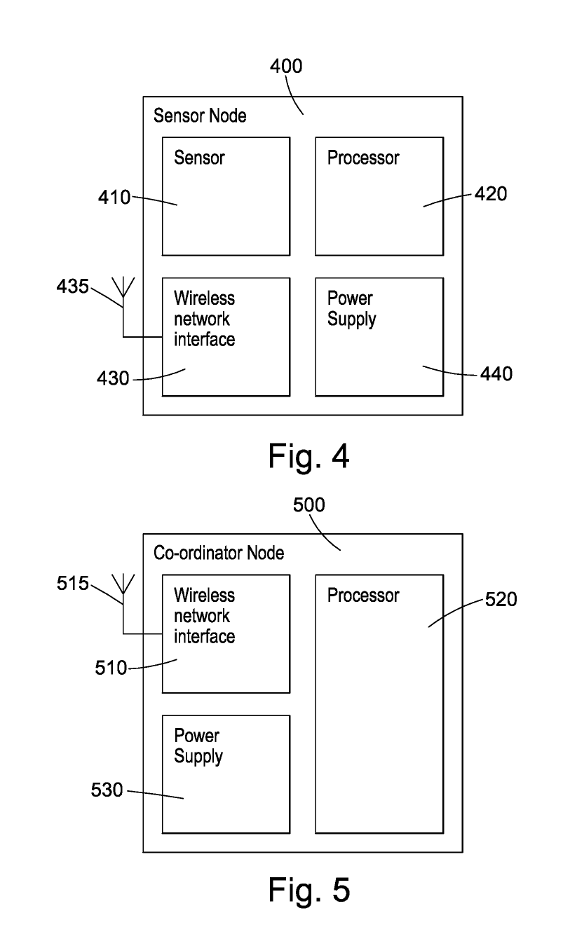

FIG. 4 shows a sensor node 400 according to an embodiment. The sensor node 400 comprises a sensor 410, a processor 420, a wireless network 430; an antenna 435; and a power supply 440. In use, the sensor node 400 is worn by a patient or implanted in the patient's body. The sensor 410 can be any type of sensor for monitoring the patient's vital signs such as a blood pressure sensor, an electro-cardiograph (ECG) for measuring heart activity, a thermometer, or a pulse oximeter for measuring blood oxygen saturation, the sensor may also be a motion sensor or accelerometer. The sensor node 400 may have a combination of sensors. The processor 420 performs processing on the sensed data and also controls the wireless network interface 430. The wireless network interface 430 allows wireless communication with a co-ordinator node. The wireless network interface 430 is coupled to the antenna 435. The wireless network interface 430 is configured to measure the received signal strength or signals received from the co-ordinator node by the antenna 435. The power supply 440 supplies power to the sensor node, and is for example a battery. It is noted that as the sensor node 400 may be implanted in a patient, or worn by a patient, it is advantageous for the size of the sensor node 400 to be minimised. Therefore, energy consumption by the node is an important consideration. This is particularly the case with implanted sensors.

FIG. 5 shows a co-ordinator node 500 according to an embodiment. The co-ordinator node comprises a wireless network interface 510; an antenna 515; a processor 520 and a power supply 530. The wireless network interface 510 allows the co-ordinator node 500 to communicate with sensor nodes via the antenna 515. The wireless network interface 510 may also allow the co-ordinator node 500 to communicate with a server which processes and stores information. The processor 520 controls the wireless network interface 510 and configures medium access control parameters of the communication between the co-ordinator node 500 and sensor nodes. The power supply 530, which is for example a battery, supplies power to the co-ordinator node. Even though the co-ordinator node 500 is worn by a patient, power consumption by the co-ordinator node is a less important issue than it is for the sensor nodes. Therefore, the majority of the function of control of the communication between the sensor nodes and the co-ordinator nodes takes place on the co-ordinator node.

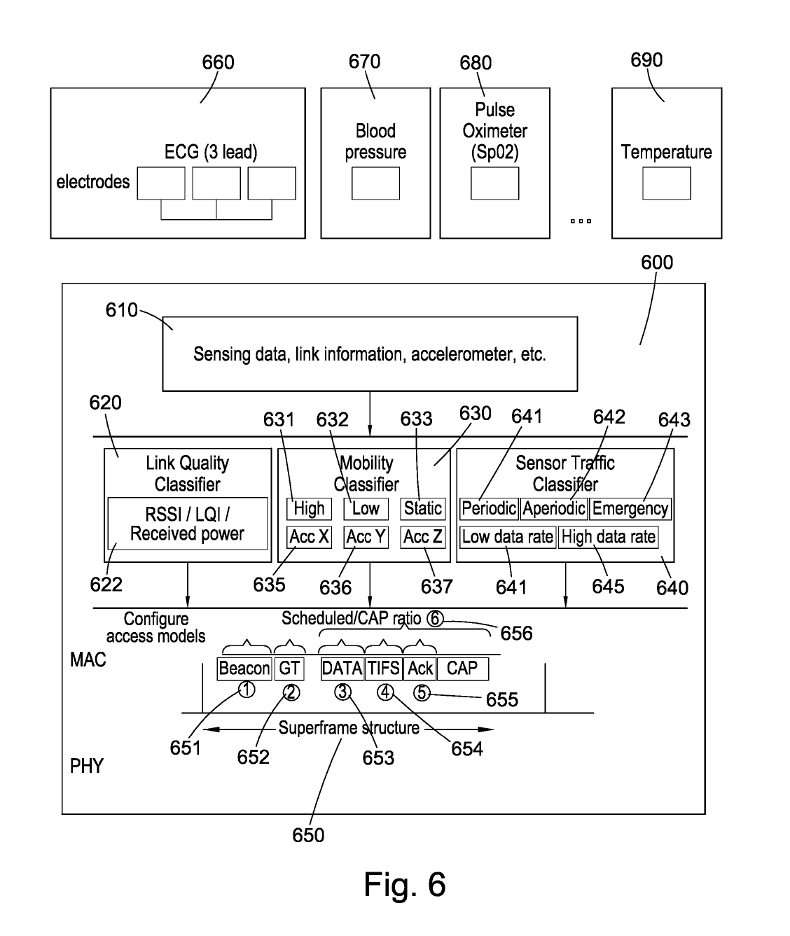

FIG. 6 shows the processing performed a co-ordinator node 600 according to an embodiment. In the embodiment shown in FIG. 6, the co-ordinator node 600 is part of a wireless body area network which comprises an electrocardiograph (ECG) sensor node 660, a blood pressure sensor node 670, a pulse oximeter sensor node 670 and a temperature sensor node 690. In addition to the vital sign sensors, the sensor nodes 660 670 680 690 may also include motion sensors.

In the coordinator node, the data 610 received from the sensor nodes is processed using a classifier based configuration process. The data 610 received from the sensor nodes includes sensed data such as ECG data, and blood pressure data, link information such as RSSI (received signal strength indications) and accelerometer data.

The co-ordinator node 600 comprises a classifying engine comprising a link quality classifier 620, a mobility classifier 630, and a sensor traffic classifier 640. The classifiers may be implemented as computer program modules running on a processor. The classifiers are used to select a configuration for medium access control superframes used by the WBAN. The link quality classifier 620 classifies the state of a link between the co-ordinator node and one of the sensor nodes according to link quality data 622 such as a received signal strength indicator (RSSI), a link quality indicator (LQI) or a received power received by the sensor node.

The mobility classifier 630 classifies the movement into one the following classifications high mobility 631; low mobility 632; or static 633. This classification is based on x-direction accelerometer data 635; y-direction accelerometer data 636 and z-direction accelerometer data 637.

Sensor Traffic Classifier 640 classifies the sensor data traffic as periodic 641; aperiodic 642 or emergency 643 and also classifies the data rate as low 644 or high 645.

Based on the results of the classification parameters of the superframe structure 650 are configured. The parameters that are configured are the beacon period 651; the guard time 652; the length of data slots 653; the interframe spacing 654; the number or time period for acknowledgements 655; and the ratio between scheduled and contention access periods 656.

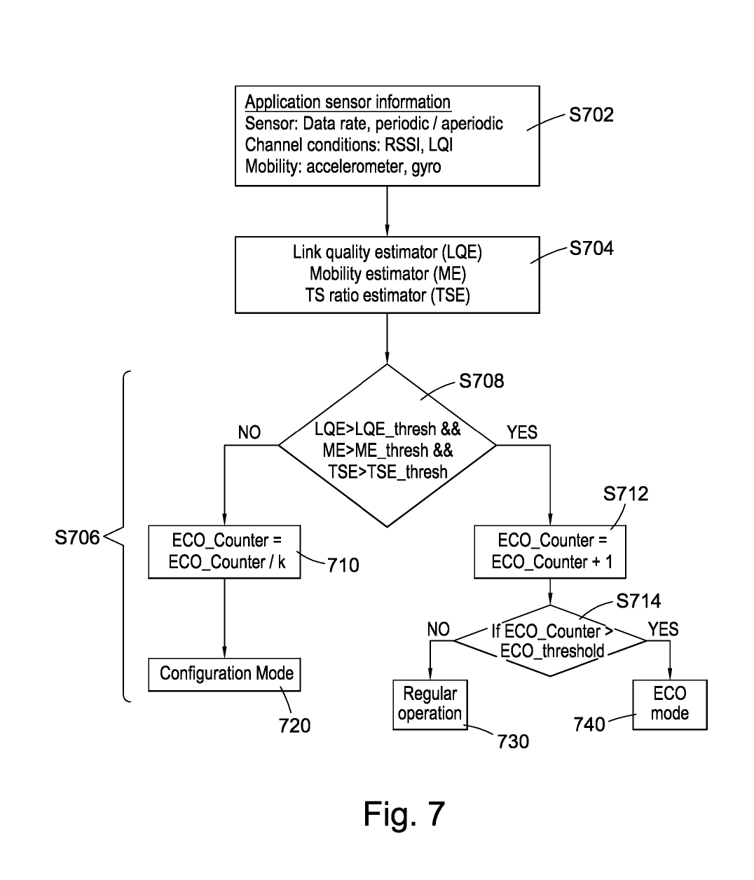

FIG. 7 shows a method of configuring superframe parameters according to an embodiment. The method comprises three main steps: an information processing an analysis step S702; an estimation step S704; and a decision and configuration step S706.

In the analysis step S702, information received from the sensor nodes is analysed. The information received includes time stamp, reference location coordinates, RSSI, and actual sensor information (such as accelerometer or gyroscope if available). Upon receiving the time-series of values from the nodes, the coordinator node determines whether a node is on a moving limb, the magnitude of RSSI fluctuations at the sensor node. The values are then compared with earlier available data and measured against the threshold to proceed to the next step only when it is warranted.

The estimation step S704 comprises (a) link quality estimation in which the extent of changes in link quality is gathered; (b) Mobility recognition and estimation and (c) sensor application data rate performance estimation, which is used to determine time slot estimation. These are described in more detail below.

Link Quality Estimation (LQE)

Link quality estimation forms a fundamental component of the WBAN communication and network protocols. Link quality estimators are based on specific radio link measurement (e.g. RSSI/LQI) and/or perceived logical connectivity information (e.g. packet reception (PRR)/loss ratio (PLR)). In body area networks, with likely scenarios of low duty cycled intermittent transmission, it is challenging to estimate the link quality. Due to the high variance, the windowed mean values are better indicators than the latest received values to determine the link state. With intermittent information, Window Mean with Exponentially Weighted Moving Average (VVM-EWMA) approach enables to capture the long-term stability and quality of each radio link. The exponential weighted approach and weight presents importance of recent measurement. The link quality estimation (LQE) of node i at time t is given by,

LQEₜⁱ=α*RSSIₘₑₐₛⁱ+α(1−α)*RSSI_{{t-1}}ⁱ+α(1−α)²*RSSI_{{t-2}}ⁱ+. . . α(1−α)^{N}*RSSI_{{t-N}}ⁱ

where α represents degree of weighting decrease between 0 and 1. A higher α discounts the older channel conditions faster. The number of samples considered can be limited (N) to last transmission period (T).

If there are other such link quality estimators available, they can also be included in the observations. For example, LQI (link quality indicator) is a measure of successfully received packets. For instance, the IEEE 802.15.4-based chipset CC2420 provides LQI based on the first eight symbols for each incoming packet. The values are usually between 50 and 110 indicating the minimum and maximum quality. Also, the LQI has higher variance than RSSI.

RSSIₜⁱ=α*RSSIₘₑₐₛⁱ+α(1−α)*RSSI_{{t-1}}ⁱ+α(1−α)²*RSSI_{{t-2}}ⁱ+. . . α(1−α)^{N}* RSSI_{{t-N}}ⁱ

LQIₜⁱ=α*LQIₘₑₐₛⁱ+α(1−α)*LQI_{{t-1}}ⁱ+α(1−α)²*LQI_{{t-2}}ⁱ+. . . α(1−α)^{N}*LQI_{{t-N}}ⁱ

The above two estimates can be normalised to common scale combined into:

LQEₜⁱ=β*RSSIₜⁱ+(1−β)*LQIₜⁱ

where β is the weighting given to RSSI.

Mobility Recognition and Estimation (ME)

The mobility/action recognition uses the on-body sensors. The sensor nodes may have accelerometers integrated into the sensor chipset. The tri-axial accelerometer sensors are tiny sensors that are used to collect acceleration of sensor nodes in different directions(x, y, z).

In an embodiment, the sensor node comprises a bi-axial gyroscope in this case, the inclination angle (θ) and azimuth angle (φ) are obtained. In an embodiment, the sensors node comprises a 9 Degree of Freedom—9DoF gyroscope then the sensor captures data via the tri-axial accelerometer, tri-axial gyro, and tri-axial magnetometer.

The sensor node i with triaxial accelerometers measures (x₁(t), y₁(t), z₁(t)) at t, and we are interested in the features extracted over N sliding windows of those segments, e.g. mean, standard deviation, root mean square, first and second derivatives. These values are subsequently transmitted to the hub coordinator.

For convenience, let us take 1-s windows. The 1-s window is moved over the signal and the mean and standard deviation corresponding to each window is calculated. If s₁, s₂, . . . , s_{N} be the standard deviations of N samples in the last transmission period with s_{N} being the latest sample , the motion estimation (ME) is given by weighted moving average (WMA) of all the N samples,

$${ME}_{t}^{i} = \frac{N * {s}_{N}^{i} + ( N - 1 ) * {s}_{\{ N - 1 \}}^{i} + ( N - 2 ) * {s}_{\{ N - 2 \}}^{i} + … 1 * {s}_{1}^{i}}{N + ( N - 1 ) + ( N - 2 ) + … + 1}$$

Time Slot Estimation (TSE)

A wide range of heterogeneous sensor and applications can be included in the WBAN. The ratio of scheduled period to the contention period can vary depending on the applications and associated traffic demand. By default, the nodes use the slot by scheduled allocation. The contention access period is set for nodes that specifically register to transmit on free-timing, that is, randomly accessing the medium to transmit. Any changes in the sensor or application require changes in the ratio—the number of scheduled allocation slots is decreased and the number of Contention access slots is increased proportionately.

The scheduled allocation provides high bandwidth efficiency. As the matter of fine resource management, the scheduled time-slot size can be adapted depending on the usage and efficiency. The coordinator monitors the actual bandwidth usage of the scheduled time slots, this can be estimated by moving-average over the utilization over N super-frames.

Let t be the current super-frame index. The coordinator estimates the scheduled timeslot utilization of sensor device i at the end of the Nth superframe as

$${TSE}_{t}^{i} = \frac{1}{N} {\sum }_{T = t - N}^{N} {u}_{T}^{i} + {v}_{T}^{i}$$

where uⁱ and vⁱ denote the amount of data transmitted to/from end device i and possible voidtime.

In step S706 the estimated parameters are run against thresholds to classify the action. The configuration features are classified into three types: configuration mode (CONF_mode), regular mode (REG_mode) and economical mode (ECO_mode). The processing of step S706 in an embodiment will now be described. In step S708, the values of LQ, ME and TSE for a sensor node are compared with thresholds.

It is noted that the comparisons with the thresholds may not take place for every link with every sensor node as the links may not be active. The hub has stored values of the thresholds. The values of the thresholds are set for example to maintain certain quality of service.

To achieve this classification objective over several superframes, the classifier maintains an ECO_Counter. The classifier adopts additive-increase multiplicative-decrease (AIMD) as appropriate feedback counter mechanism.

If one of the estimated parameters is below a respective threshold, the method moves to step S710 in which the ECO_Counter is divided by a constant k. This is the multiplicative decrease part of the feedback mechanism. Following step S710, the network is placed in Configuration Mode 720.

If all of the parameters are determined to be above the respective thresholds in step S708, the method moves to step S712. In step S712, the ECO_Counter is incremented by 1. This is the additive increase part of the feedback mechanism. Following step S714, the ECO_Counter is compared with a threshold. If the ECO_Counter is above the threshold, the network is placed in ECO Mode 740. If the ECO_Counter is below the threshold, the network is placed in Regular Operation mode 730.

Based on the above estimated values and decision, the superframe parameters are configured according to the mode in which the network is placed. Synchronization is a critical issue in scheduled access TDMA MAC where the nodes and the hub are precisely synchronized in order to transmit/receive packets successfully. Since the nodes and hub/coordinator have their own clocks, they are difficult to synchronize when they wake up after a long period of sleep. This synchronization issue is tackled and the resilience of synchronization is improved by configuring the parameters—beacon, GT, IFS, and ACK.

3(a) Beacon Width Adaptation:

Ideally, the sensor node's listening interval is expected to be short. Thus, with keeping the listening period minimal for the nodes, the hub/coordinator increases the beacon period in CONF_mode.

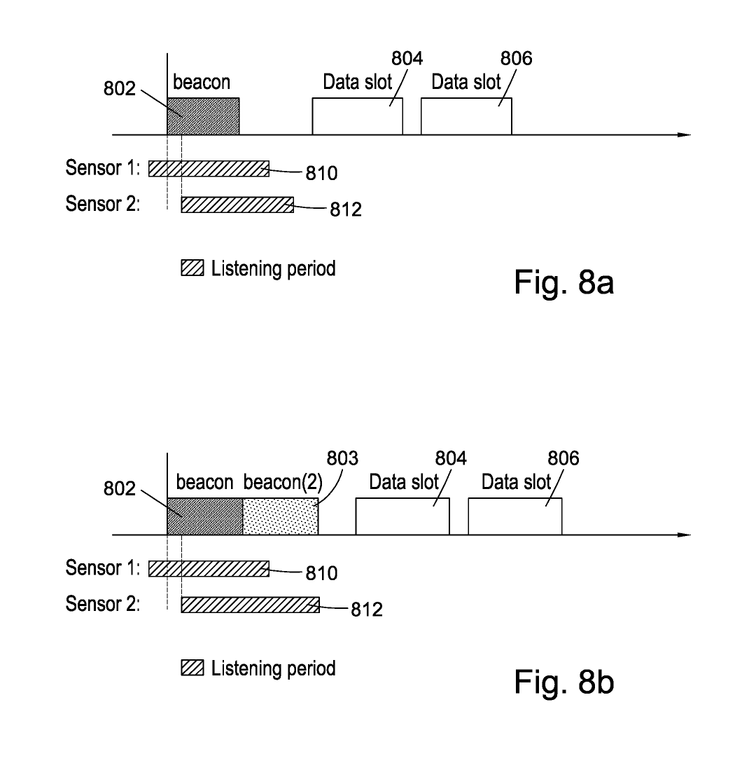

FIG. 8a shows the beacon width in regular operation in an embodiment. A beacon 802 of beacon width T_{beacon} is transmitted at the beginning of a superframe by the co-ordinator node. The beacon 802 is followed by a first data slot 804 and a second data slot 806. As shown in FIG. 8a, the listening period 810 of a first sensor node (Sensor 1) overlaps with the beginning of the beacon 802, therefore the first sensor node receives the beacon 802. The listening period 812 of a second sensor node (Sensor 2) starts after the beginning of the beacon 802. Therefore the second sensor node does not receive all of the beacon packets, therefore it is considered not to have received the beacon 802.

FIG. 8b shows the beacon width in configuration mode in an embodiment. A first beacon 802 of beacon width T_{beacon} and a second beacon 803 of beacon width T_{beacon} are transmitted at the beginning of a superframe by the co-ordinator node. The beacons 802 & 803 are followed by a first data slot 804 and a second data slot 806. As shown in FIG. 8b, the listening period 812 of the second sensor node (Sensor 2) overlaps with the beginning of the second beacon 803, therefore both the first sensor node receive the beacons. The first sensor node receives the first beacon 802 and the second sensor node receives the second beacon 803.

Exploiting the good channel conditions, ECO_mode allows the network to skip beacons.

Thus the beacon width is set according to the mode as follows:

$${T}_{beacon} = \{ \begin{matrix}0.5 * {T}_{beacon} , & ECO\_mode \\ {T}_{beacon} , & REG\_mode \\ 2 * {T}_{beacon} , & CONF\_mode\end{matrix}$$

3(b) Guard Time Adaptation:

Maintaining synchronisation of devices while sleeping through beacons can be achieved by adaptive guard time. Guard times are inserted to alleviate any clock drift. For packet transmission the clock drift and synchronization issue could be small, but considering worst-case scenario, guard time is increased in CONF_mode.

$${T}_{GT} = \{ \begin{matrix}{T}_{GT} , & ECO\_mode \\ {T}_{GT} , & REG\_mode \\ 2 * {T}_{GT} , & CONF\_mode\end{matrix}$$

3(c) Interframe Space Adaptation:

Inter frame space is the amount of time necessary to process the received packet by the physical layer (PHY). Transmitted frames are followed by an interframe space (IFS) period. The length of IFS depends on the size of the frame that has just been transmitted. Following the T_{IFS} period, the radio would transmit (TX) and then try to go back to receiving (RX). Some radios may need more time (to finish the TX->RX transition). For this reason, T_{IFS} is configured as follows.

$${T}_{IFS} = \{ \begin{matrix}{T}_{IFS} , & ECO\_mode \\ {T}_{IFS} , & REG\_mode \\ 2 * {T}_{IFS} , & CONF\_mode\end{matrix}$$

3(d) Timeslot Adaptation:

The scheduled allocation provides high bandwidth efficiency. As the matter of fine resource management, the scheduled time-slot size can be adapted depending on the usage and efficiency. Based on the TSE and LQE, the coordinator could vary the TS allocated, thus varying the packet size.

$${T}_{TS} = \{ \begin{matrix}2 * {T}_{TS} , & ECO\_mode \\ {T}_{TS} , & REG\_mode \\ 0.5 * {T}_{TS} , & CONF\_mode\end{matrix}$$

3(e) Acknowledgement Adaptation:

Mobile and long sleeping nodes require clock drifting resilience. The resilience of synchronization is improved by increased number of acknowledgments. Increasing the number of acknowledgments shows improved utilization with very limited impact on energy efficiency.

$${T}_{ACK} = \{ \begin{matrix}0.5 * {T}_{ACK} , & ECO\_mode \\ {T}_{ACK} , & REG\_mode \\ 2 * {T}_{ACK} , & CONF\_mode\end{matrix}$$

3(f) Active Period Ratio

The ratio of scheduled period to the contention period can vary depending on the applications and associated traffic demand.

The scheduled mode or contention access is selected during the start-up stage, so the initial ratio is selected during the node registration/start-up stage. Then, during operation stage, the hub may determine how much of the nodes/packets are going to be scheduled and how much of it is going to be contention based. That ratio alteration happens during the operation stage in this algorithm.

In some low data rate applications or underperforming nodes, it is noted that the performance is improved by switching to contention access. Based on the TSE and LQE, the number of scheduled allocation slots is decreased if the number of contention access slots is increased, and vice versa, by the same amount. For example, in a superframe with 16 timeslot active frame, if the Scheduled versus Contention ratio (denoted by “:”) are set to 15:1, 12:4, 10:6, 6:10, 4:12, 2:14 then the configured access selects the ratio that performs the best in given condition.

In an embodiment the active period T_{active} may be varied based on the mode as follows:

$${T}_{active} = \{ \begin{matrix}0.5 * {T}_{active} , & ECO\_mode \\ {T}_{active} , & REG\_mode \\ 2 * {T}_{active} , & CONF\_mode\end{matrix}$$

In embodiments, the classifier-based configuration methods provides sufficient flexibility to the TDMA schedule. Also, in ECO_mode, the mechanism exploits the favourable conditions to save energy for the sensor nodes.

The wide range of sensors (e.g. ECG, EEG, pulse oximeter etc.,) have diverse data rate and QoS requirements. The mechanism described in the embodiments estimates the application data rate performances and link quality and configures various superframe and access mode parameters—thus delivering high quality of service (QoS).

There are several advantages to the proposed embodiments have several advantages: The classifier-based configuration method provides flexibility to the tightly synchronized scheduled access superframe structure. The proposed mechanism makes use of the available intermittent and statistically driven information. The advantages include removing the outliers during estimating but still reacting to the changes in the radio environment. This mechanism adapts the diverse data rate and QoS requirements of the wide range of sensors attached. Embodiments allow the exploitation of favourable conditions and thus is highly energy efficient.

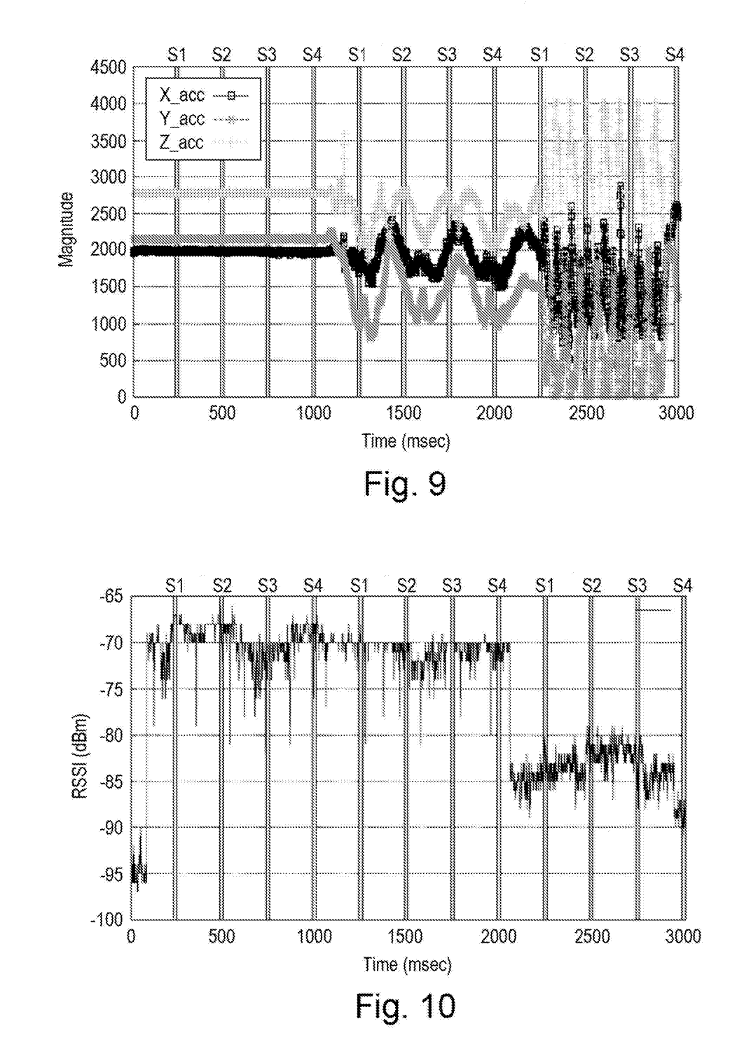

FIG. 9 shows the sampling of accelerometer values for motion estimation (ME) step in an embodiment. In the shown figure, 4 samples (N=4) labelled as S1 to S4 with sampling period (ΔW) of 20 ms every 250 ms is used in the computation of ME as described above in the section titled ‘Mobility recognition and estimation (ME)’. As shown in FIG. 9, initially, there is little or no variation in acceleration measured by the sensors. After approximately 1100 ms, a small change magnitude occurs, this is first measured in the second S1 sampling period which occurs at 1250 ms. Then at 2250 ms a large change fluctuation occurs. This is first detected in the third window S2 at 2500 ms.

FIG. 10 illustrates the sampling of RSSI values toward computation of LQE. The RSSI values are sampled over the 20 ms periods labelled S1 to S4. As shown in FIGS. 9 and 10, the sampling periods are relatively small compared to the periods in which no sampling occurs. However, the network is able to adapt to changes in conditions even though the sampling periods are relatively short.

The specific embodiments are presented schematically. The reader will appreciate that the detailed implementation of each embodiment can be achieved in a number of ways.

For instance, a dedicated hardware implementation could be designed and built. On the other hand, a processor could be configured with a computer program, such as delivered either by way of a storage medium (e.g. a magnetic, optical or solid state memory based device) or by way of a computer receivable signal (e.g. a download of a full program or a “patch” update to an existing program) to implement the management unit described above in relation to the embodiments. Besides these two positions, a multi-function hardware device, such as a DSP, a FPGA or the like, could be configured by configuration instructions.

Whilst certain embodiments have been described, these embodiments have been presented by way of example only, and are not intended to limit the scope of the inventions. Indeed, the novel devices, methods and systems described herein may be embodied in a variety of other forms; furthermore, various omissions, substitutions and changes in the form of the devices, methods and systems described herein may be made without departing from the spirit of the inventions. The accompanying claims and their equivalents are intended to cover such forms or modifications as would fall within the scope and spirit of the inventions.

1. A method of medium access control in a co-ordinator node of a body area network, the body area network comprising the co-ordinator node and a plurality of sensor nodes, each of the sensor nodes comprising a sensor and being configured to wirelessly communicate with the co-ordinator node in time slots defined by a plurality of medium access control superframes, the method comprising
receiving at the co-ordinator node, a signal from a first sensor node of the plurality of sensor nodes, the signal comprising an indication of a measured quantity at the first sensor node, the measured quantity being indicative of an attribute of a wireless link of the body area network between the first sensor node and the co-ordinator node;
estimating an attribute of the link using the indication; and
configuring parameters of a medium access control superframe using the estimated attribute.

2. A method according to claim 1, wherein the attribute of the link is movement of the first sensor node.

3. A method according to claim 2, wherein the parameters of a medium access control superframe comprise at least one of beacon width; guard time; interframe space; and acknowledgement period.

4. A method according to claim 1, comprising receiving a plurality of indications of the measured quantity from the first sensor node; and determining a weighted moving average value from the received values wherein the attribute of the link is estimated using the weighted moving average value.

5. A method according to claim 1, wherein configuring parameters of a medium access control superframe using the estimated attribute comprises comparing the attribute of the link with a threshold; selecting a mode based on the result of the comparison and selecting values of parameters of the medium access control superframe according to the selected mode.

6. A method according to claim 5 wherein the mode is selected using additive increase multiplicative decrease feedback.

7. A method according to claim 1, wherein the attribute of the link is an active period time slot.

8. A method according to claim 6, wherein configuring parameters of the medium access control superframe comprises configuring the ratio of active and inactive period of the superframe based on the active period time slot.

9. A method according to claim 1 wherein the parameters of the medium access control superframe comprise timing parameters.

10. A computer readable carrier medium carrying computer readable instructions which when executed on a processor cause the processor to carry out a method according to claim 1.

11. A co-ordinator node for a wireless body area network comprising
an antenna configured to receive signals from a plurality of sensor nodes of the wireless body area network, the signals comprising a first signal from a first sensor node of the plurality of sensor nodes, the first signal comprising an indication of a measured quantity at the first sensor node, the measured quantity being indicative of a attribute of a link of the wireless body area network between the first sensor node and the co-ordinator node;
a processor operable to
estimate an attribute of the link using the indication; and
configure parameters of a medium access control superframe using the estimated attribute.

12. A co-ordinator node according to claim 11, wherein the attribute of the link is movement of the first sensor node.

13. A co-ordinator node according to claim 12, wherein the parameters of a medium access control superframe comprise at least one of beacon width; guard time; interframe space; and acknowledgement period.

14. A co-ordinator node according to claim 11, wherein the processor comprises a classifier operable to classify the state of the link based on the estimated attribute of the link.

15. A co-ordinator node according to claim 14, wherein the classifier is operable to classify the state of the link y comparing the estimated attribute with a threshold.

16. A co-ordinator node according to claim 11, wherein the processor is operable to configure the parameters of the medium access control superframe by selecting a mode based on the estimated attribute.

17. A wireless body area network comprising a co-ordinator node according to claim 11 and
at least one sensor node comprising a sensor operable measure a quantity; and an antenna operable to transmit a first signal comprising an indication of the measured quantity.

18. A wireless body area network according to claim 17, wherein the sensor is an accelerometer and the measured quantity is movement.
